(19) United States
(12) Patent Application Publication
(10) Pub. No.: US 2026/0097144 A1
(43) Pub. Date: Apr. 9, 2026

(54) ADJUSTABLE AIR FRESHENER DEVICE

(71) Applicant: Ron Halliday, Las Vegas, NV (US)

(72) Inventor: Ron Halliday, Las Vegas, NV (US)

(21) Appl. No.: 18/905,261

(22) Filed: Oct. 3, 2024

(51) Int. Cl. A61L 9/12

(52) U.S. Cl. CPC A61L 9/12

(57) ABSTRACT

An adjustable air freshener device includes a rear panel and a plurality of grips each attached to the rear panel. A front panel is positionable on the rear panel and each of the plurality of grips releasably engages the front panel for removably retaining the front panel on the rear panel thereby enabling the front panel and the rear panel to define an encasement. The front panel has a plurality of vents to pass air into and out of the encasement. A vent cover is slidably attached to the front panel to modulate the amount of air that can pass through the plurality of vents between a maximum amount and a minimum amount. An air freshener is positionable between the rear panel and the front panel. The air freshener is infused with a chemical scent thereby enabling the chemical scent to enhance the odor of ambient air.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to air freshener devices and more particularly pertains to a new air freshener device for enhancing the odor of ambient air. The device includes a rear panel which has a plurality of grips and a front panel that is releasably matable to the plurality of grips for attaching the front panel to the rear panel. The front panel includes a plurality of vents and the device includes a vent cover that is movably integrated into the front panel for modulating the amount of air that can pass through the vents. The device includes an air freshener that is positionable between the front panel and the rear panel and the air freshener is infused with a chemical scent that can pass through the vents to enhance the odor of ambient air.

### (2) Description of Related Art Including Information Disclosed Under 37 CFR 1.97 and 1.98

The prior art relates to air freshener devices including: an air freshener device that includes a container which contains a fluid fragrance and a foraminous lid which is attachable to the container and a permeable barrier integrated into the foraminous lid to enable a fragrance to pass through the permeable barrier; an air freshener holder device that includes a holder that has a channel for insertably receiving an air freshener and a pair of wings each angling upwardly from the holder which can attach to a rear view mirror of a vehicle; an air freshener device that includes a container for containing a solid air freshener and a lid that is releasably attachable to the container and attachment for securing the container to a surface. In no instance does the prior art disclose an air freshener device that includes a rear panel and a plurality of grips attached to the rear panel and a front panel that is matable to the grips and a plurality of vents extending through the front panel and a vent cover movably attached to the front panel for modulating the amount of air that passes through the vents and an air freshener that is positionable between the front panel and the rear panel.

### BRIEF SUMMARY OF THE INVENTION

An embodiment of the disclosure meets the needs presented above by generally comprising a rear panel and a plurality of grips each attached to the rear panel. A front panel is positionable on the rear panel and each of the plurality of grips releasably engages the front panel for removably retaining the front panel on the rear panel thereby enabling the front panel and the rear panel to define an encasement. The front panel has a plurality of vents to pass air into and out of the encasement. A vent cover is slidably attached to the front panel to modulate the amount of air that can pass through the plurality of vents between a maximum amount and a minimum amount. An air freshener is positionable between the rear panel and the front panel. The air freshener is infused with a chemical scent thereby enabling the chemical scent to enhance the odor of ambient air.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an exploded perspective view of an adjustable air freshener device according to an embodiment of the disclosure.

FIG. 2 is a left side view of a front panel of an embodiment of the disclosure.

FIG. 3 is a back view of a front panel of an embodiment of the disclosure.

FIG. 4 is a front view of a rear panel of an embodiment of the disclosure.

FIG. 5 is a left side view of a rear panel of an embodiment of the disclosure.

FIG. 6 is a back view of a rear panel of an embodiment of the disclosure.

FIG. 7 is an exploded perspective view of an embodiment of the disclosure.

FIG. 8 is a left side view of an embodiment of the disclosure.

FIG. 9 is a left side cut-away view of an embodiment of the disclosure.

FIG. 10 is a perspective in-use view of an embodiment of the disclosure.

FIG. 11 is a front view of an alternative embodiment of the disclosure.

FIG. 12 is an in-use view of an alternative embodiment of the disclosure.

FIG. 13 is a perspective view of an alternative embodiment of the disclosure.

FIG. 14 is a front view of a front panel of an embodiment of the disclosure.

### DETAILED DESCRIPTION OF THE INVENTION

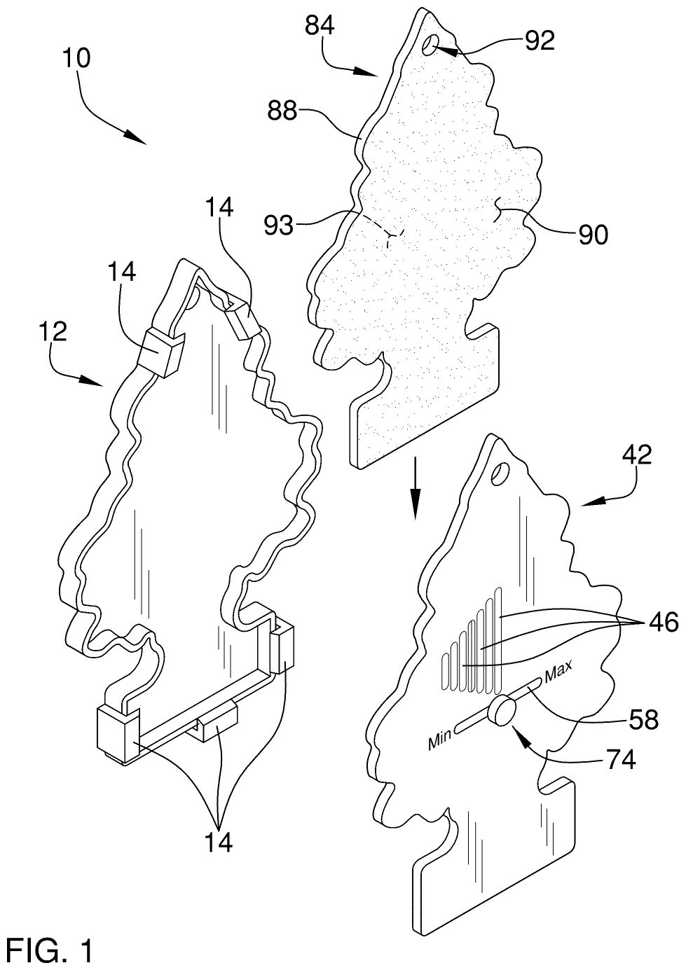

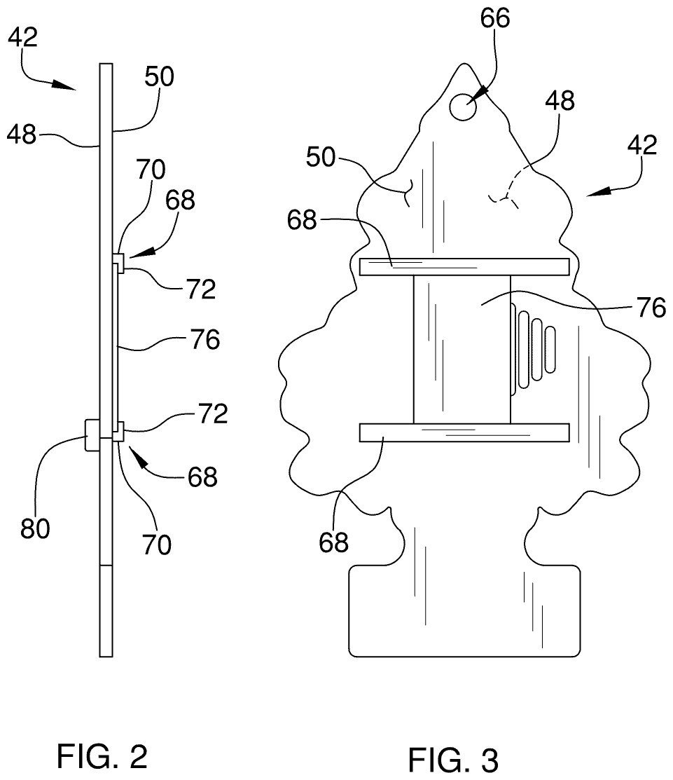

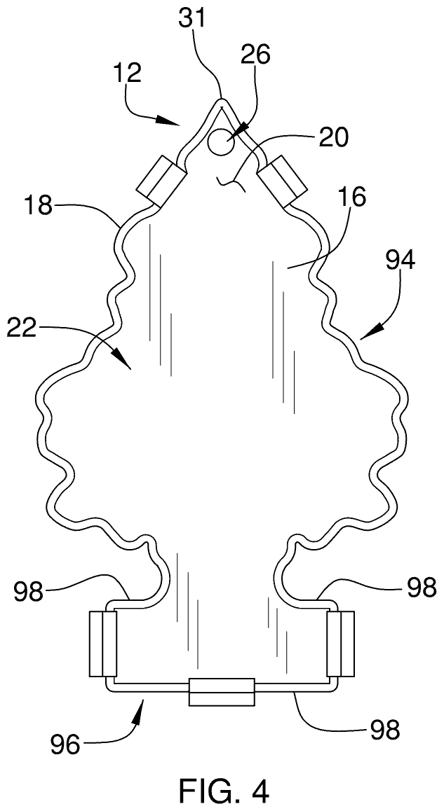

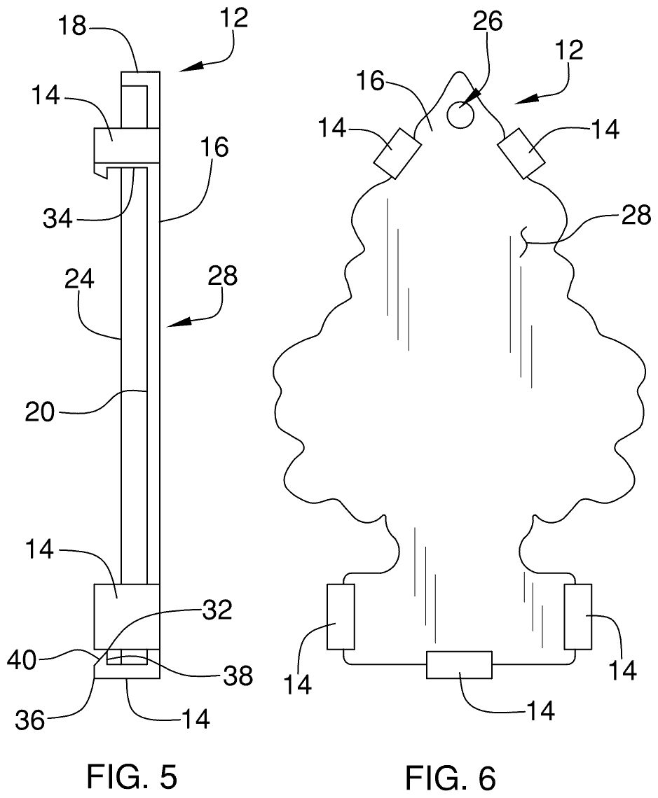

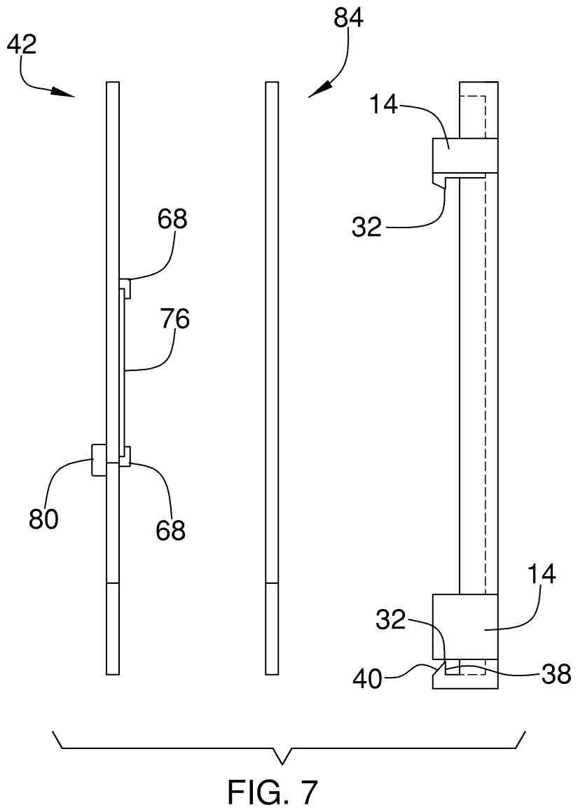

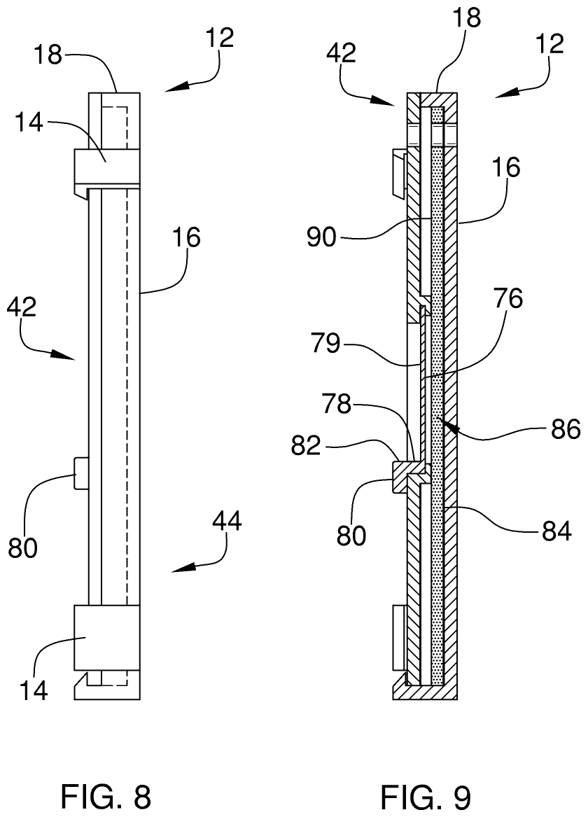

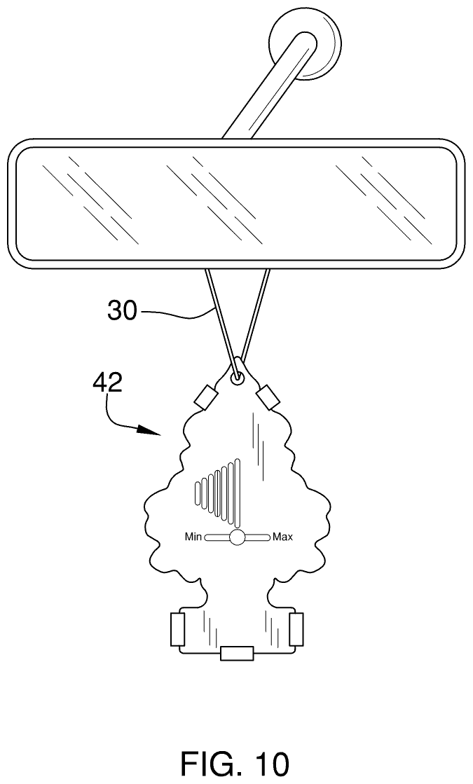

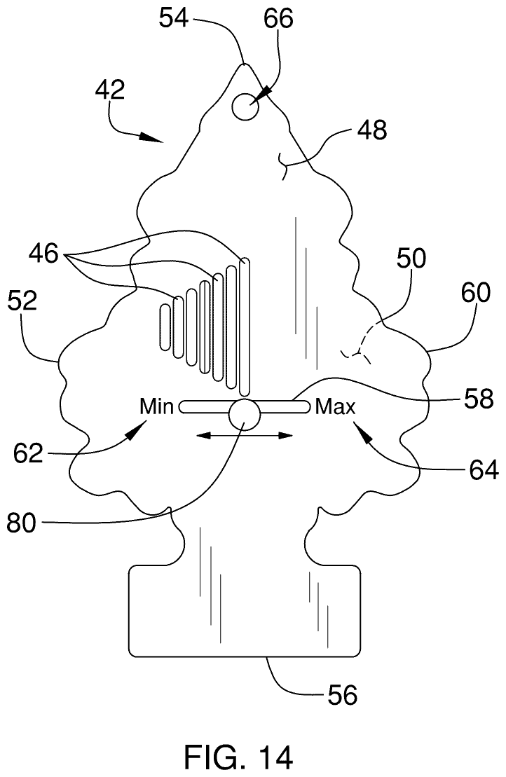

With reference now to the drawings, and in particular to FIGS. 1 through 14 thereof, a new air freshener device embodying the principles and concepts of an embodiment of the disclosure and generally designated by the reference numeral 10 will be described.

As best illustrated in FIGS. 1 through 14, the adjustable air freshener device 10 generally comprises a rear panel 12 which has a plurality of grips 14 each attached to the rear panel 12. The grips 14 are spaced apart from each other and are distributed around the rear panel 12. The rear panel 12 has a rear wall 16 and a perimeter wall 18 extending away from a front surface 20 of the rear wall 16 such that the perimeter wall 18 defines a space 22 in the rear panel 12. Each of the plurality of grips 14 is integrated into the perimeter wall 18 having each of the plurality of grips 14 extending beyond a distal edge 24 of the perimeter wall 18 with respect to the front surface 20 of the rear wall 16.

The rear panel 12 has a hole 26 extending through the front surface 20 of the rear wall 16 and a back surface 28 of the rear wall 16. The hole 26 accommodates a suspension 30 thereby enabling the rear panel 12 to be suspended from the suspension 30 and the hole 26 is positioned proximate an upper end 31 of the rear panel 12. Each of the plurality of grips 14 is comprised of a resiliently flexible material, including but not being limited to plastic or rubber, to enable the grips 14 to be flexed outwardly on the perimeter wall 18. The suspension 30 may comprise a string, for example, which is suspended from a rear view mirror in a vehicle, for example.

Each of the plurality of grips 14 includes a finger 32 which extends away from an inwardly facing surface 34 of a respective grip 14 such that the finger 32 on each of the plurality of grips 14 extends toward the space 22 defined in the rear panel 12. The finger 32 on each of the plurality of grips 14 is aligned with a distal end 36 of the respective grip 14. Additionally, the finger 32 on each of the plurality of grips 14 has a back side 38 which is perpendicularly oriented with the respective grip 14. The finger 32 on each of the plurality of grips 14 has a front side 40 which slopes downwardly between the distal end 36 of the respective grip 14 and the back side 38.

A front panel 42 is positionable on the rear panel 12 having each of the plurality of grips 14 releasably engaging the front panel 42 for removably retaining the front panel 42 on the rear panel 12. In this way the front panel 42 and the rear panel 12 define an encasement 44. The front panel 42 has a plurality of vents 46 each extending through the front panel 42 to pass air into and out of the encasement 44 defined by the front panel 42 and the rear panel 12. Each of the plurality of vents 46 extends through a forward surface 48 and a rear surface 50 of the front panel 42 and each of the plurality of vents 46 is elongated along a vertical axis of the front panel 42. Furthermore, the plurality of vents 46 is evenly spaced apart from each other and is distributed along a lateral axis of the front panel 42. The plurality of vents 46 extends from a center line of the front panel 42 toward a first lateral edge 52 of the front panel 42. The plurality of vents 46 range in length between a maximum length and a minimum length such that the plurality of vents 46 defines a triangular shape which extends from the center line toward the first lateral edge 52. Additionally, the plurality of vents 46 is centrally located between a top end 54 and a bottom end 56 of the front panel 42.

The front panel 42 has a slot 58 extending through the forward surface 48 and the rear surface 50 of the front panel 42 and the slot 58 is elongated along the lateral axis of the front panel 42. The slot 58 is positioned beneath the plurality of vents 46 and the slot 58 is centrally located between the first lateral edge 52 and a second lateral edge 60 of the front panel 42. The front panel 42 has first indicia 62 which are applied to the forward surface 48 and the first indicia 62 is positioned between the slot 58 and the first lateral edge 52. Furthermore, the first indicia 62 comprise the word “min”. The front panel 42 has second indicia 64 which are applied to the forward surface 48 and the second indicia 64 are positioned between the slot 58 and the second lateral edge 60. Furthermore, the second indicia 64 comprise the word “max”. The front panel 42 has a hole 66 extending through the forward surface 48 and the rear surface 50 of the front panel 42 and the hole 66 in the front panel 42 is positioned proximate the top end 54 of the front panel 42.

The rear surface 50 of the front panel 42 slides along the front side 40 of the finger 32 on each of the plurality of grips 14 on the rear panel 12 until the front panel 42 is seated beneath the back side 38 of the finger 32 on each of the plurality of grips 14. In this way the rear surface 50 of the front panel 42 is retained against the distal edge 24 of the perimeter wall 18 of the rear panel 12 thereby enabling the front panel 42 to cover the space 22 defined in the rear panel 12. The hole 58 in the front panel 42 is aligned with the hole 26 in the rear panel 12 when the front panel 42 is mated to the rear panel 12 to accommodate the suspension 30. The front panel 42 has pair of rails 68 which each has a first section 70 that is coupled to and extends away from the rear surface 50 of the front panel 42 and a second section 72 which is spaced from and is oriented parallel to the rear surface 50. Each of the pair of rails 68 is oriented to extend along the lateral axis of the front panel 42. Additionally, a respective one of the pair of rails 68 is located above the plurality of vents 46 and a respective one of the pair of rails 68 is located below the plurality of vents 46.

A vent cover 74 is slidably attached to the front panel 42 and the vent cover 74 is aligned with the plurality of vents 46 in the front panel 42. The vent cover 74 is slidable between a fully closed position having the vent cover 74 completely covering the plurality of vents 46 and a fully open position having the vent cover 74 being displaced from the plurality of vents 46. In this way the vent cover 74 can modulate the amount of air that can pass through the plurality of vents 46 between a maximum amount and a minimum amount. The vent cover 74 includes a vent panel 76 which is positioned between the second section 72 of each of the pair of rails 68 on the front panel 42 and the rear surface 50 of the front panel 42 such that the vent panel 76 is slidable along the pair of rails 68. The vent cover 74 includes a stem 78 which is coupled to and extends away from a front surface 79 of the vent panel 76 and the stem 78 extends through the slot 58 in the front panel 42. The vent cover 74 includes a knob 80 which is attached to a distal end 82 of the stem 78 such that the knob 80 is spaced from the forward surface 48 of the front panel 42 thereby enabling the knob 80 to be manipulated for urging the vent cover 74 between the fully open position and the fully closed position.

An air freshener 84 is positionable against the rear panel 12 before the front panel 42 is positioned on the rear panel 12 such that the air freshener 84 is retained in the encasement 44 defined by the front panel 42 and the rear panel 12. The air freshener 84 is infused with a chemical scent 86 and the chemical scent 86 is released through the plurality of vents 46 in the front panel 42 thereby enabling the chemical scent 86 to enhance the odor of ambient air. The chemical scent 86 may be a floral scent, a spice scent or any other generally pleasing scent. The air freshener 84 is positionable in the space 22 defined by the perimeter wall 18 of the rear panel 12 such that a back surface 28 of the air freshener 84 rests against the front surface 20 of the rear panel 12.

The air freshener 84 has a perimeter edge 88 which is structured to conform to a shape defined by the perimeter wall 18 of the rear panel 12 thereby inhibiting the air freshener 84 from freely moving in the space 22. The second section 72 of each of the pair of rails 68 on the rear surface 50 of the front panel 42 rests against a front surface 90 of the air freshener 84 when the front panel 42 is mated to the rear panel 12 thereby securing the air freshener 84 in the space 22 defined in the rear panel 12. The air freshener 84 has a hole 92 extending through the front surface 90 of the air freshener 84 and a back surface 93 of the air freshener 84. The hole 92 in the air freshener 84 is aligned with the hole 58 in the front panel 42 and the hole 26 in the rear panel 12 when the air freshener 84 is positioned in the space 22 to accommodate the suspension 30. Additionally, the vent panel 76 modulates the amount of chemical scent 86 that can pass through the plurality of vents 46 in the front panel 42 thereby enabling the vent panel 76 to adjust the amount of the chemical scent 86 that is released through the plurality of vents 46 between a maximum amount and a minimum amount.

The perimeter wall 18 of the rear panel 12 is structured to have an upper portion 94 which undulates and a lower portion 96 which has a plurality of perpendicularly oriented sides 98 such that the perimeter wall 18 defines the outline of a pine tree. Furthermore, the perimeter wall 18 of the back panel may be structured to define a variety of different ornamental shapes. Each of the first lateral edge 52 and the second lateral edge 60 of the front panel 42 are structured to undulate in a manner which corresponds to the upper portion 94 of the perimeter wall 18 of the rear panel 12 such that the front panel 42 defines the outline of a pine tree. Additionally, each of the first lateral edge 52 and the second lateral edge 60 of the front panel 42 may be structured to define a variety of ornamental shapes which corresponds to the ornamental shape defined by the rear panel 12. Furthermore, the perimeter edge 88 of the air freshener 84 is structured to match the ornamental shape defined by each of the front panel 42 and the rear panel 12.

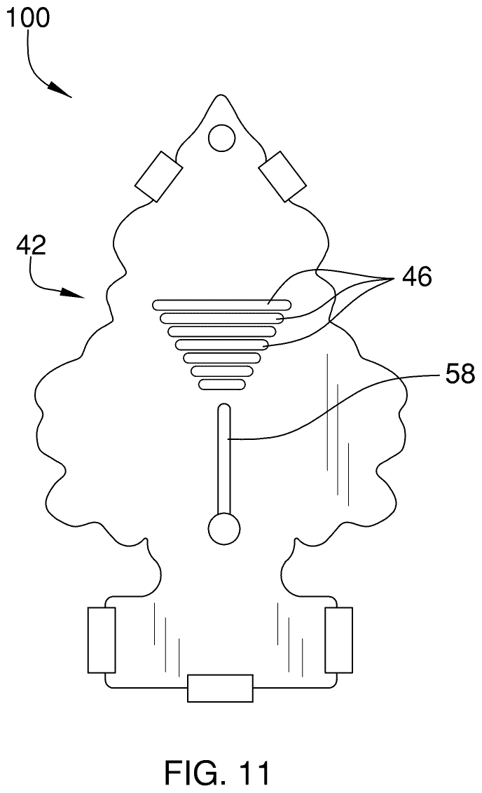

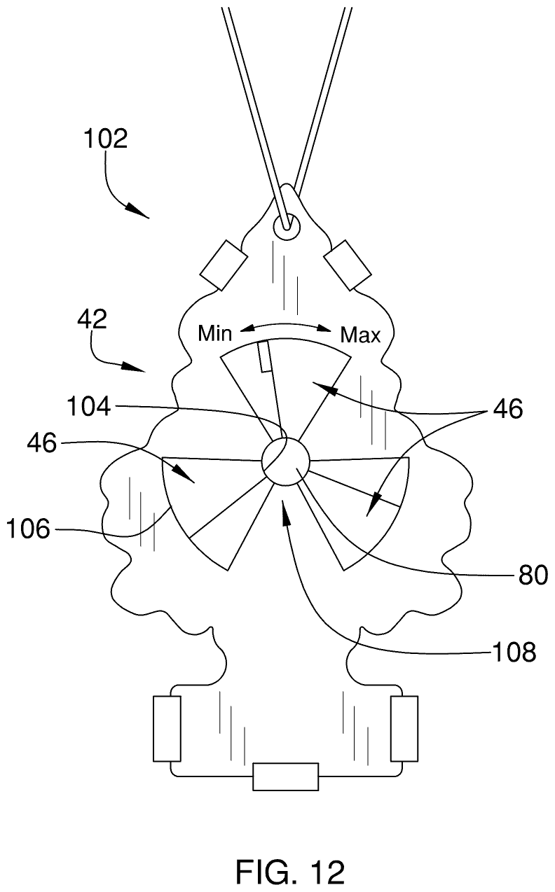

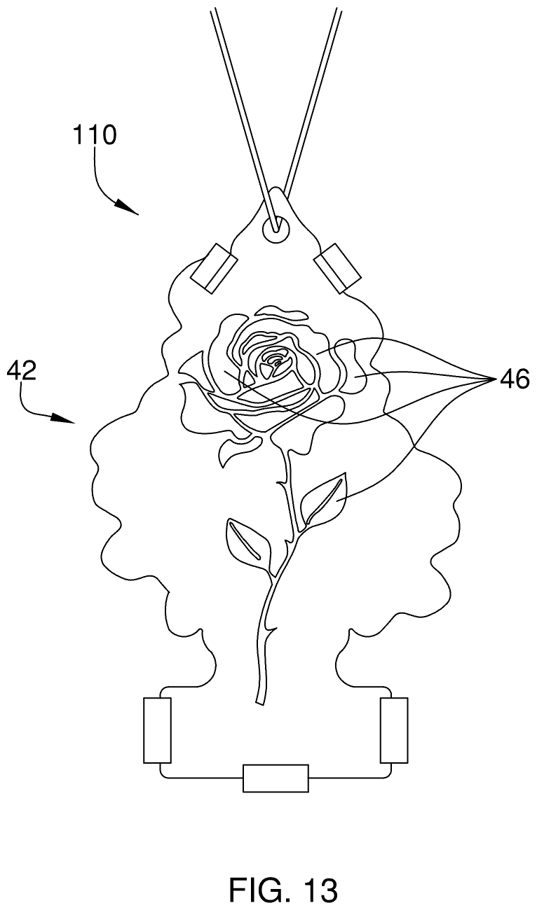

In an alternative embodiment 100 shown in FIG. 11, the plurality of vents 46 are evenly spaced apart from each other and are distributed along a vertical axis of the front panel 42. Additionally, the slot 58 is elongated along a vertical axis of the front panel 42 and the slot 58 is positioned beneath the plurality of vents 46. In an alternative embodiment 102 shown in FIG. 12, each of the plurality of vents 46 has a first end 104 and a second end 106 and each of the plurality of vents 46 widens between the first end 104 and the second end 106 such that each of the plurality of vents 46 has a wedge shape. Additionally, the plurality of vents 46 is spaced apart from each other and is distributed around a central point 108 having the first end 104 of each of the plurality of vents 46 being directed toward the central point 108. Furthermore, the knob 80 is aligned with the central point 108 thereby enabling the vent cover 74 to be rotated with the knob 80 for opening or closing the plurality of vents 46. In an alternative embodiment 110 shown in FIG. 13, each of the plurality of vents 46 is structured to collectively define the ornamental appearance of a rose.

In use, the air freshener 84 is placed in the space 22 defined in the rear panel 12 and the front panel 42 is mated to the rear panel 12 to retain the air freshener 84 between the front panel 42 and the rear panel 12. The knob 80 is manipulated to move the vent panel 76 to either completely cover the vents 46 or to expose one or more of the vents 46, depending on a user's preference. In this way the degree of chemical scent 86 that passes through the plurality of vents 46 can be controlled. The suspension 30 is extended through the hole 26 in the rear panel 12 and the hole 92 in the air freshener 84 and the hole 92 in the front panel 42 to enable the rear panel 12 and the air freshener 84 and the front panel 42 to be suspended from the suspension 30. The front panel 42 is unmated from the rear panel 12 when the chemical scent 86 in the air freshener 84 has been depleted and the air freshener 84 is replaced with a new air freshener.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An adjustable air freshener device for controlling the amount of an air freshener that is released into ambient air, said device comprising:
a rear panel having a plurality of grips each being attached to said rear panel, said grips being spaced apart from each other and being distributed around said rear panel;
a front panel being positionable on said rear panel having each of said plurality of grips releasably engaging said front panel for removably retaining said front panel on said rear panel thereby enabling said front panel and said rear panel to define an encasement, said front panel having a plurality of vents each extending through said front panel wherein each of said plurality of vents is configured to pass air into and out of said encasement defined by said front panel and said rear panel;
a vent cover being slidably attached to said front panel, said vent cover being aligned with said plurality of vents in said front panel, said vent cover being slidable between a fully closed position having said vent cover completely covering said plurality of vents and a fully open position having said vent cover being displaced from said plurality of vents wherein said vent cover is configured to modulate the amount of air that can pass through said plurality of vents between a maximum amount and a minimum amount; and
an air freshener being positionable against said rear panel before said front panel is positioned on said rear panel such that said air freshener is retained in said encasement defined by said front panel and said rear panel, said air freshener being infused with a chemical scent wherein said chemical scent is configured to be released through said plurality of vents in said front panel thereby enabling said chemical scent to enhance the odor of ambient air.

2. The adjustable air freshener device according to claim 1, wherein:
said rear panel has a rear wall and a perimeter wall extending away from a front surface of said rear wall such that said perimeter wall defines a space in said rear panel;
each of said plurality of grips is integrated into said perimeter wall having each of said plurality of grips extending beyond a distal edge of said perimeter wall with respect to said front surface of said rear wall;
each of said plurality of grips includes a finger which extends away from an inwardly facing surface of a respective grip such that said finger on each of said plurality of grips extends toward said space defined in said rear panel;
said finger on each of said plurality of grips is aligned with a distal end of said respective grip;
said finger on each of said plurality of grips has a back side being perpendicularly oriented with said respective grip;
said finger on each of said plurality of grips has a front side which slopes downwardly between said distal end of said respective grip and said back side;
said rear panel has a hole extending through said front surface of said rear wall and a back surface of said rear wall wherein said hole is configured to accommodate a suspension thereby enabling said rear panel to be suspended from the suspension; and
said hole is positioned proximately an upper end of said rear panel.

3. The adjustable air freshener device according to claim 1, wherein:
each of said plurality of vents extends through a forward surface and a rear surface of said front panel;
each of said plurality of vents is elongated along a vertical axis of said front panel;
said plurality of vents is evenly spaced apart from each other and is distributed along a lateral axis of said front panel;
said plurality of vents extends from a center line of said front panel toward a first lateral edge of said front panel;
said plurality of vents range in length between a maximum length and a minimum length such that said plurality of vents defines a triangular shape which extends from said center line toward said first lateral edge;
said plurality of vents is centrally located between a top end and a bottom end of said front panel;
said front panel has a slot extending through said forward surface and said rear surface of said front panel;
said slot is elongated along said lateral axis of said front panel;
said slot is positioned beneath said plurality of vents; and
said slot is centrally located between said first lateral edge and a second lateral edge of said front panel.

4. The adjustable air freshener device according to claim 3, wherein:
said front panel has first indicia being applied to said forward surface;
said first indicia are positioned between said slot and said first lateral edge;
said first indicia comprise the word “min”;
said front panel has second indicia being applied to said forward surface;
said second indicia are positioned between said slot and said second lateral edge; and
said second indicia comprise the word “max”.

5. The adjustable air freshener device according to claim 1, wherein:
said front panel has a hole extending through said forward surface and said rear surface of said front panel;
said hole in said front panel is positioned proximate said top end of said front panel; and
said hole in said front panel is aligned with a hole in said rear panel when said front panel is mated to said rear panel wherein said hole in said front panel is configured to accommodate a suspension.

6. The adjustable air freshener device according to claim 2, wherein a rear surface of said front panel slides along said front side of said finger on each of said plurality of grips on said rear panel until said front panel is seated beneath said back side of said finger on each of said plurality of grips for retaining said rear surface of said front panel against said distal edge of said perimeter wall of said rear panel thereby enabling said front panel to cover said space defined in said rear panel.

7. The adjustable air freshener device according to claim 3, wherein:
said front panel has pair of rails each having a first section being coupled to and extending away from said rear surface of said front panel and a second section being spaced from and being oriented parallel to said rear surface;
each of said pair of rails is oriented to extend along said lateral axis of said front panel;
a respective one of said pair of rails is located above said plurality of vents; and
a respective one of said pair of rails is located below said plurality of vents.

8. The adjustable air freshener device according to claim 7, wherein:
said vent cover includes a vent panel being positioned between said second section of each of said pair of rails on said front panel and said rear surface of said front panel such that said vent panel is slidable along said pair of rails;
said vent cover includes a stem being coupled to and extending away from a front surface of said vent panel;
said stem extends through said slot in said front panel; and
said vent cover includes a knob being attached to a distal end of said stem such that said knob is spaced from said forward surface of said front panel thereby enabling said knob to be manipulated for urging said vent cover between said fully open position and said fully closed position.

9. The adjustable air freshener device according to claim 7, wherein:
said air freshener is positionable in said space defined by said perimeter wall of said rear panel such that a back surface of said air freshener rests against said front surface of said rear panel;
said air freshener has a perimeter edge which is structured to conform to a shape defined by said perimeter wall of said rear panel thereby inhibiting said air freshener from freely moving in said space; and
said second section of each of said pair of rails on said rear surface of said front panel rests against a front surface of said air freshener when said front panel is mated to said rear panel thereby securing said air freshener in said space defined in said rear panel.

10. The adjustable air freshener device according to claim 8, wherein:
said front panel has a hole extending through said front panel;
said rear panel has a hole extending through said rear panel;
said air freshener has a hole extending through said front surface of said air freshener and said back surface of said air freshener;
said hole in said air freshener is aligned with said hole in said front panel and said hole in said rear panel when said air freshener is positioned in a space defined in said rear panel wherein said hole in said air freshener is configured to accommodate the suspension; and
said vent panel modulates the amount of chemical scent that can pass through said plurality of vents in said front panel thereby enabling said vent panel to adjust the amount of said chemical scent that is released through said plurality of vents between a maximum amount and a minimum amount.

11. An adjustable air freshener device for controlling the amount of an air freshener that is released into ambient air, said device comprising:
a rear panel having a plurality of grips each being attached to said rear panel, said grips being spaced apart from each other and being distributed around said rear panel, said rear panel having a rear wall and a perimeter wall extending away from a front surface of said rear wall such that said perimeter wall defines a space in said rear panel, each of said plurality of grips being integrated into said perimeter wall having each of said plurality of grips extending beyond a distal edge of said perimeter wall with respect to said front surface of said rear wall, each of said plurality of grips including a finger which extends away from an inwardly facing surface of a respective grip such that said finger on each of said plurality of grips extends toward said space defined in said rear panel, said finger on each of said plurality of grips being aligned with a distal end of said respective grip, said finger on each of said plurality of grips having a back side being perpendicularly oriented with said respective grip, said finger on each of said plurality of grips having a front side which slopes downwardly between said distal end of said respective grip and said back side, said rear panel having a hole extending through said front surface of said rear wall and a back surface of said rear wall wherein said hole is configured to accommodate a suspension thereby enabling said rear panel to be suspended from the suspension, said hole being positioned proximate an upper end of said rear panel;
a front panel being positionable on said rear panel having each of said plurality of grips releasably engaging said front panel for removably retaining said front panel on said rear panel thereby enabling said front panel and said rear panel to define an encasement, said front panel having a plurality of vents each extending through said front panel wherein each of said plurality of vents is configured to pass air into and out of said encasement defined by said front panel and said rear panel, each of said plurality of vents extending through a forward surface and a rear surface of said front panel, each of said plurality of vents being elongated along a vertical axis of said front panel, said plurality of vents being evenly spaced apart from each other and being distributed along a lateral axis of said front panel, said plurality of vents extending from a center line of said front panel toward a first lateral edge of said front panel, said plurality of vents ranging in length between a maximum length and a minimum length such that said plurality of vents defines a triangular shape which extends from said center line toward said first lateral edge, said plurality of vents being centrally located between a top end and a bottom end of said front panel, said front panel having a slot extending through said forward surface and said rear surface of said front panel, said slot being elongated along said lateral axis of said front panel, said slot being positioned beneath said plurality of vents, said slot being centrally located between said first lateral edge and a second lateral edge of said front panel, said front panel having first indicia being applied to said forward surface, said first indicia being positioned between said slot and said first lateral edge, said first indicia comprising the word “min”, said front panel having second indicia being applied to said forward surface, said second indicia being positioned between said slot and said second lateral edge, said second indicia comprising the word “max”, said front panel having a hole extending through said forward surface and said rear surface of said front panel, said hole in said front panel being positioned proximate said top end of said front panel, said rear surface of said front panel sliding along said front side of said finger on each of said plurality of grips on said rear panel until said front panel is seated beneath said back side of said finger on each of said plurality of grips for retaining said rear surface of said front panel against said distal edge of said perimeter wall of said rear panel thereby enabling said front panel to cover said space defined in said rear panel, said hole in said front panel being aligned with said hole in said rear panel when said front panel is mated to said rear panel wherein said hole in said front panel is configured to accommodate the suspension, said front panel having pair of rails each having a first section being coupled to and extending away from said rear surface of said front panel and a second section being spaced from and being oriented parallel to said rear surface, each of said pair of rails being oriented to extend along said lateral axis of said front panel, a respective one of said pair of rails being located above said plurality of vents, a respective one of said pair of rails being located below said plurality of vents;
a vent cover being slidably attached to said front panel, said vent cover being aligned with said plurality of vents in said front panel, said vent cover being slidable between a fully closed position having said vent cover completely covering said plurality of vents and a fully open position having said vent cover being displaced from said plurality of vents wherein said vent cover is configured to modulate the amount of air that can pass through said plurality of vents between a maximum amount and a minimum amount, said vent cover comprising:
a vent panel being positioned between said second section of each of said pair of rails on said front panel and said rear surface of said front panel such that said vent panel is slidable along said pair of rails;
a stem being coupled to and extending away from a front surface of said vent panel, said stem extending through said slot in said front panel; and
a knob being attached to a distal end of said stem such that said knob is spaced from said forward surface of said front panel thereby enabling said knob to be manipulated for urging said vent cover between said fully open position and said fully closed position; and
an air freshener being positionable against said rear panel before said front panel is positioned on said rear panel such that said air freshener is retained in said encasement defined by said front panel and said rear panel, said air freshener being infused with a chemical scent wherein said chemical scent is configured to be released through said plurality of vents in said front panel thereby enabling said chemical scent to enhance the odor of ambient air, said air freshener being positionable in said space defined by said perimeter wall of said rear panel such that a back surface of said air freshener rests against said front surface of said rear panel, said air freshener having a perimeter edge which is structured to conform to a shape defined by said perimeter wall of said rear panel thereby inhibiting said air freshener from freely moving in said space, said second section of each of said pair of rails on said rear surface of said front panel resting against a front surface of said air freshener when said front panel is mated to said rear panel thereby securing said air freshener in said space defined in said rear panel, said air freshener having a hole extending through said front surface of said air freshener and said back surface of said air freshener, said hole in said air freshener being aligned with said hole in said front panel and said hole in said rear panel when said air freshener is positioned in said space wherein said hole in said air freshener is configured to accommodate the suspension, said vent panel modulating the amount of chemical scent that can pass through said plurality of vents in said front panel thereby enabling said vent panel to adjust the amount of said chemical scent that is released through said plurality of vents between a maximum amount and a minimum amount.

12. The adjustable air freshener device according to claim 1, wherein:
said plurality of vents are evenly spaced apart from each other and are distributed along a vertical axis of said front panel;
said front panel has a slot extending through a forward surface and a rear surface of said front panel;
said slot is elongated along a vertical axis of said front panel;
said slot is positioned beneath said plurality of vents; and
said vent cover includes a knob being aligned with said slot such that said knob is slidably along said slot for opening or closing said plurality of vents.

13. The adjustable air freshener device according to claim 1, wherein:
each of said plurality of vents has a first end and a second end;
each of said plurality of vents widens between said first end and said second end such that each of said plurality of vents has a wedge shape;
said plurality of vents is spaced apart from each other and is distributed around a central point having said first end of each of said plurality of vents being directed toward said central point; and
said vent cover includes a knob being aligned with said central point thereby enabling said vent cover to be rotated with said knob for opening or closing said plurality of vents.

14. The adjustable air freshener device according to claim 1, wherein each of said plurality of vents is structured to collectively define the ornamental appearance of a rose.
